(19) United States
(12) Patent Application Publication
(10) Pub. No.: US 2016/0106515 A1
(43) Pub. Date: Apr. 21, 2016

(54) Determining the Positional Information of Characteristic Points of a Leg for Osteotomy

(71) Applicant: BRAINLAB AG, Feldkirchen (DE)

(72) Inventors: Sabine Kling, Unterschleibheim (DE); Luise Poitzsch, Au i. d. Hallertau (DE); Melanie Stulpe, Munchen (DE); Mario Schubert, Poing (DE); Christianäß Brack, Neusäß (DE); Timo Neubauer, Grasbrunn-Neukeferloh (DE)

(21) Appl. No.: 14/895,019

(22) PCT Filed: Dec. 12, 2013
(86) PCT No.: PCT/EP2013/076305

§ 371 Date: Dec. 1, 2015

(30) Foreign Application Priority Data
Aug. 13, 2013 (EP) PCT/EP2013/066867
Aug. 14, 2013 (EP) PCT/EP2013/067006

(51) Int. Cl. A61B 5/00; A61B 5/11; A61B 17/60

(52) U.S. Cl. CPC A61B 19/5244; A61B 5/6887; A61B 5/1114; A61B 5/1127; A61B 19/54; A61B 19/46; A61B 2019/5248; A61B 2019/5255; A61B 2019/5265; A61B 2019/5268; A61B 2019/467; A61B 2019/5287; A61B 2019/5483; A61B 2019/5491

(57) ABSTRACT

A data processing method for determining the positional information of characteristic points of a leg, the method comprising the following steps performed by a computer: a) acquiring, by detecting via a hand-held device a stationary reference (R3) and at least one further information, at least four different positions of the femur (F), wherein the pelvis within which the femur (F) can turn is stationary with respect to the stationary reference (R3) and the femur (F) is in a different position each time a positional information value of the femur (F) is acquired; b) determining from the at least four different acquired positional information values of the femur (F) the position of the center of rotation (COR) of the femoral head in relation to a femur reference (R1, R4); c) acquiring a femur information by detecting via a hand-held device a femur reference (R1), and at least one further information; d) determining from the femur information and the at least one further information acquired in step c) the distal end point of the femur axis and the proximal end point of the tibia axis at least in relation to the femur reference (R1); and e) determining the distal end point of the tibia axis by acquiring via a hand-held device the positional information of an ankle reference (R2) being at the distal end point of the tibia axis.

### FIELD OF THE INVENTION

The present invention is directed to the determination of positional information of characteristic points of a leg, preferably to be used for osteotomy surgery on femur and tibia.

### BACKGROUND OF THE INVENTION

An osteotomy is a surgical operation whereby a bone is cut to shorten, lengthen, or change its alignment. When performing navigated closed or open wedge tibial osteotomies, it is necessary to register the patient anatomy, requiring the invasive attachment of reference arrays to the patient's bone. This, however, increases the risk of infection and secondary bone fractures. Large navigation systems having expensive stereo cameras are required together with a pointer device for anatomy registration purposes.

### PRIOR ART

WO 2004/049941 A1 discloses a non-invasive method for determining the articular point of a joint. The method uses a surgical navigation system having a stereoscopic camera which tracks the positions of infrared markers attached to appendages on either side of the joint. A moveable marker is used to palpate known landmarks on the appendages to determine their positions. The appendages are moved to determine the trajectories of the landmarks relative to the joint. Positional information from the camera is fed to a data processing system with software which uses the positional information and trajectories to mathematically determine the position of the joint articular point according to the laws of kinematics.

US 2002/0198451 A1 discloses surgical navigation systems and processes for tracking anatomy, instrumentation, and references, and rendering images and data related to them in connection with surgical operations, particularly high tibial osteotomy.

US 2007/0118140 A1 discloses a method and an apparatus for navigating a cutting tool during orthopedic surgery using a localization system.

WO 95/00075 A1 discloses a method and an apparatus for locating functional structures of the lower leg during knee implant surgery by determining the location of the weight bearing axis (WBA), determining the preferred location of the WBA, determining the preferred location of the knee implant, and guiding the instruments used in the shaping of bone required to locate the implant.

WO 2006/119387 A2 discloses a system and a method for determining tibial rotation. The system includes a first fiducial, a second fiducial, a position and orientation sensor, a computer, and a monitor. The first fiducial is connected to a first part and the second fiducial is connected to a second part. The position and orientation sensor tracks the first fiducial and the second fiducial. The computer receives data from the position and orientation sensor and processes the data to identify a first axis of the first part and a second axis of the second part and constructs a reference plane through the second axis and orthogonal to the first axis.

Known systems for tibia osteotomy are for example Brainlab's VectorVision®, OrthoPilot® (Aesculap) or SurgiGate (Medivision).

### OBJECTS OF THE INVENTION

It is an object of the present invention to determine the positional information of characteristic points of a leg, preferably the end points of the mechanical femur axis and the mechanical tibia axis, which can then be used for osteotomy surgery on femur and tibia. It is a further object to determine the varus/valgus angle or the flexion angle of a leg using the mentioned positional information.

These objects are achieved by the subject-matter of the independent claims. The dependent claims are directed to advantageous embodiments. Different advantageous features can be combined in accordance with the invention wherever technically expedient and feasible. A feature of one embodiment which is functionally identical or similar to a feature of another embodiment can in particular replace the latter feature. A feature of an embodiment which supplements the function of another embodiment can in particular be added to the other embodiment.

### DEFINITIONS

Hereinafter an explanation and a definition of terms used to describe the invention is given.

### Data Processing Method

The method in accordance with the invention is in particular a data processing method. The data processing method is preferably performed using technical means, in particular a computer. The data processing method is preferably constituted to be executed by or on a computer and in particular is executed by or on the computer. In particular, all the steps or merely some of the steps (i.e. less than the total number of steps) of the method in accordance with the invention can be executed by a computer. The computer in particular comprises a processor and a memory in order to process the data, in particular electronically and/or optically. The calculating steps described are in particular performed by a computer. Determining steps or calculating steps are in particular steps of determining data within the framework of the technical data processing method, in particular within the framework of a program. A computer is in particular any kind of data processing device, in particular electronic data processing device. A computer can be a device which is generally thought of as such, for example desktop PCs, notebooks, netbooks, etc., but can also be any programmable apparatus, such as for example a mobile phone or an embedded processor. A computer can in particular comprise a system (network) of “sub-computers”, wherein each sub-computer represents a computer in its own right. The term “computer” includes a cloud computer, in particular a cloud server. The term “cloud computer” includes a cloud computer system which in particular comprises a system of at least one cloud computer and in particular a plurality of operatively interconnected cloud computers such as a server farm. Such a cloud computer is preferably connected to a wide area network such as the world wide web (WWW) and located in a so-called cloud of computers which are all connected to the world wide web. Such an infrastructure is used for “cloud computing”, which describes computation, software, data access and storage services which do not require the end user to know the physical location and/or configuration of the computer delivering a specific service. In particular, the term “cloud” is used in this respect as a metaphor for the Internet (world wide web). In particular, the cloud provides computing infrastructure as a service (IaaS). The cloud computer can function as a virtual host for an operating system and/or data processing application which is used to execute the method of the invention. The cloud computer is for example an elastic compute cloud (EC2) as provided by Amazon Web Services™. A computer in particular comprises interfaces in order to receive or output data and/or perform an analogue-to-digital conversion. The data are in particular data which represent physical properties and/or which are generated from technical signals. The technical signals are in particular generated by means of (technical) detection devices (such as for example devices for detecting marker devices) and/or (technical) analytical devices (such as for example devices for performing imaging methods), wherein the technical signals are in particular electrical or optical signals. The technical signals in particular represent the data received or outputted by the computer. The computer is preferably operatively coupled to a display device which allows information outputted by the computer to be displayed, for example to a user. One example of a display device is an augmented reality device (also referred to as augmented reality glasses) which can be used as “goggles” for navigating. A specific example of such augmented reality glasses is Google Glass (a trademark of Google, Inc.). An augmented reality device can be used both to input information into the computer by user interaction and to display information outputted by the computer.

### Acquiring Data

The expression “acquiring data” in particular encompasses (within the framework of a data processing method) the scenario in which the data are determined by the data processing method or program. Determining data in particular encompasses measuring physical quantities and transforming the measured values into data, in particular digital data, and/or computing the data by means of a computer and in particular within the framework of the method in accordance with the invention. The meaning of “acquiring data” also in particular encompasses the scenario in which the data are received or retrieved by the data processing method or program, for example from another program, a previous method step or a data storage medium, in particular for further processing by the data processing method or program. The expression “acquiring data” can therefore also for example mean waiting to receive data and/or receiving the data. The received data can for example be inputted via an interface. The expression “acquiring data” can also mean that the data processing method or program performs steps in order to (actively) receive or retrieve the data from a data source, for instance a data storage medium (such as for example a ROM, RAM, database, hard drive, etc.), or via the interface (for instance, from another computer or a network). The data can be made “ready for use” by performing an additional step before the acquiring step. In accordance with this additional step, the data are generated in order to be acquired. The data are in particular detected or captured (for example by an analytical device). Alternatively or additionally, the data are inputted in accordance with the additional step, for instance via interfaces. The data generated can in particular be inputted (for instance into the computer). In accordance with the additional step (which precedes the acquiring step), the data can also be provided by performing the additional step of storing the data in a data storage medium (such as for example a ROM, RAM, CD and/or hard drive), such that they are ready for use within the framework of the method or program in accordance with the invention. The step of “acquiring data” can therefore also involve commanding a device to obtain and/or provide the data to be acquired. In particular, the acquiring step does not involve an invasive step which would represent a substantial physical interference with the body, requiring professional medical expertise to be carried out and entailing a substantial health risk even when carried out with the required professional care and expertise. In particular, the step of acquiring data, in particular determining data, does not involve a surgical step and in particular does not involve a step of treating a human or animal body using surgery or therapy. In order to distinguish the different data used by the present method, the data are denoted (i.e. referred to) as “XY data” and the like and are defined in terms of the information which they describe, which is then preferably referred to as “XY information” and the like.

### Computer Program

Within the framework of the invention, computer program elements can be embodied by hardware and/or software (this includes firmware, resident software, micro-code, etc.). Within the framework of the invention, computer program elements can take the form of a computer program product which can be embodied by a computer-usable, in particular computer-readable data storage medium comprising computer-usable, in particular computer-readable program instructions, “code” or a “computer program” embodied in said data storage medium for use on or in connection with the instruction-executing system. Such a system can be a computer; a computer can be a data processing device comprising means for executing the computer program elements and/or the program in accordance with the invention, in particular a data processing device comprising a digital processor (central processing unit or CPU) which executes the computer program elements, and optionally a volatile memory (in particular a random access memory or RAM) for storing data used for and/or produced by executing the computer program elements. Within the framework of the present invention, a computer-usable, in particular computer-readable data storage medium can be any data storage medium which can include, store, communicate, propagate or transport the program for use on or in connection with the instruction-executing system, apparatus or device. The computer-usable, in particular computer-readable data storage medium can for example be, but is not limited to, an electronic, magnetic, optical, electromagnetic, infrared or semiconductor system, apparatus or device or a medium of propagation such as for example the Internet. The computer-usable or computer-readable data storage medium could even for example be paper or another suitable medium onto which the program is printed, since the program could be electronically captured, for example by optically scanning the paper or other suitable medium, and then compiled, interpreted or otherwise processed in a suitable manner. The data storage medium is preferably a non-volatile data storage medium. The computer program product and any software and/or hardware described here form the various means for performing the functions of the invention in the example embodiments. The computer and/or data processing device can in particular include a guidance information device which includes means for outputting guidance information. The guidance information can be outputted, for example to a user, visually by a visual indicating means (for example, a monitor and/or a lamp) and/or acoustically by an acoustic indicating means (for example, a loudspeaker and/or a digital speech output device) and/or tactilely by a tactile indicating means (for example, a vibrating element or a vibration element incorporated into an instrument). For the purpose of this document, a computer is a technical computer which in particular comprises technical, in particular tangible components, in particular mechanical and/or electronic components. Any device mentioned as such in this document is a technical and in particular tangible device.

### Computer

The method in accordance with the invention is preferably at least partly executed by a computer, i.e. all the steps or merely some of the steps (i.e. less than the total number of steps) of the method in accordance with the invention can be executed by a computer.

### Marker

It is the function of a marker to be detected by a marker detection device (for example, a camera or an ultrasound receiver or analytical devices such as CT or MRI) in such a way that its spatial position (i.e. its spatial location and/or alignment) can be ascertained. The detection device is in particular part of a navigation system. The markers can be active markers. An active marker can for example emit electromagnetic radiation and/or waves which can be in the infrared, visible and/or ultraviolet spectral range. The marker can also however be passive, i.e. can for example reflect electromagnetic radiation in the infrared, visible and/or ultraviolet spectral range or can block x-ray radiation. To this end, the marker can be provided with a surface which has corresponding reflective properties or can be made of metal in order to block the x-ray radiation. It is also possible for a marker to reflect and/or emit electromagnetic radiation and/or waves in the radio frequency range or at ultrasound wavelengths. A marker preferably has a spherical and/or spheroid shape and can therefore be referred to as a marker sphere; markers can however also exhibit a cornered, for example cubic, shape.

### Marker Device

A marker device can for example be a reference star or a pointer or a single marker or a plurality of (individual) markers which are then preferably in a predetermined spatial relationship. A marker device comprises one, two, three or more markers, wherein two or more such markers are in a predetermined spatial relationship. This predetermined spatial relationship is in particular known to a navigation system and is for example stored in a computer of the navigation system.

### Reference Star

A “reference star” refers to a device with a number of markers, advantageously three markers, attached to it, wherein the markers are (in particular detachably) attached to the reference star such that they are stationary, thus providing a known (and advantageously fixed) position of the markers relative to each other. The position of the markers relative to each other can be individually different for each reference star used within the framework of a surgical navigation method, in order to enable the corresponding reference star to be identified by a surgical navigation system on the basis of the position of its markers relative to each other. It is therefore also then possible for the objects (for example, instruments and/or parts of a body) to which the reference star is attached to be identified and/or differentiated accordingly. In a surgical navigation method, the reference star serves to attach a plurality of markers to an object (for example, a bone or a medical instrument) in order to be able to detect the position of the object (i.e. its spatial location and/or alignment). Such a reference star in particular features a way of being attached to the object (for example, a clamp and/or a thread) and/or a holding element which ensures a distance between the markers and the object (in particular in order to assist the visibility of the markers to a marker detection device) and/or marker holders which are mechanically connected to the holding element and which the markers can be attached to.

### Marker Holder

A marker holder is understood to mean an attaching device for an individual marker which serves to attach the marker to an instrument, a part of the body and/or a holding element of a reference star, wherein it can be attached such that it is stationary and advantageously such that it can be detached. A marker holder can for example be rod-shaped and/or cylindrical. A fastening device (such as for instance a latching mechanism) for the marker device can be provided at the end of the marker holder facing the marker and assists in placing the marker device on the marker holder in a force fit and/or positive fit.

### Navigation System

The present invention is also directed to a navigation system for computer-assisted surgery. This navigation system preferably comprises the aforementioned computer for processing the data provided in accordance with the data processing method as described in any one of the preceding embodiments. The navigation system preferably comprises a detection device for detecting the position of the detection points which represent the main points and auxiliary points, in order to generate detection signals and to supply the generated detection signals to the computer, such that the computer can determine the absolute main point data and absolute auxiliary point data on the basis of the detection signals received. In this way, the absolute point data can be provided to the computer. The navigation system also preferably comprises a user interface for receiving the calculation results from the computer (for example, the position of the main plane, the position of the auxiliary plane and/or the position of the standard plane). The user interface provides the received data to the user as information. Examples of a user interface include a display device such as a monitor, or a loudspeaker. The user interface can use any kind of indication signal (for example a visual signal, an audio signal and/or a vibration signal). An example of a display device is an augmented reality device (also called augmented reality glasses) which may be used as goggles for navigating. A specific example of such augmented reality glasses is Google Glass (trademark of Google Inc.). An augmented reality device may be used to both input information into the computer of the navigation system by user interaction and to display information outputted by that computer.

### Pointer

A pointer is a rod which comprises one or more—advantageously, two—markers fastened to it and which can be used to measure off individual co-ordinates, in particular spatial co-ordinates (i.e. three-dimensional co-ordinates), on a part of the body within the framework of a morphing method, wherein a user guides the pointer (in particular, a part of the pointer which has a defined and advantageously fixed position with respect to the at least one marker attached to the pointer) to the position corresponding to the co-ordinates, such that the position of the pointer can be determined by using a surgical navigation system to detect the marker on the pointer. The relative location between the markers of the pointer and the part of the pointer used to measure off co-ordinates (in particular, the tip of the pointer) is in particular known. The surgical navigation system then enables the location (of the three-dimensional co-ordinates) to be assigned to a predetermined body structure, wherein the assignment can be made automatically or by user intervention.

### SUMMARY OF THE INVENTION

The present invention is directed to a data processing method for determining the positional information of characteristic points of a leg, such as the end points of the mechanical femur axis being on one end the center of rotation of the femoral head and on the opposed end of the femur axis the distal femur axis end point, both end points defining the positional orientation of the mechanical femur axis, and such as the proximal tibia axis end point and the distal tibia end point, both end points being on opposed ends and defining the position and orientation of the mechanical tibia axis. The distal femur axis end point and the proximal tibia axis end point can be treated as being the same points. In a healthy leg, the mechanical femur axis and the mechanical tibia axis are approximately in line with each other (0° varus/valgus) when the leg is in a neutral position or full extension. The method is performed by a computer and comprises the following steps:

a) a stationary reference, such as for example an arrangement of markers or a reference star having, at least while acquiring the positional information for the data processing method, a fixed positional relationship with respect to the pelvis or pivoting point around which the leg is moved, is detected. At least one further information (described below) is acquired as well. The acquiring step is performed at least four different times while the femur is in four different positions. For example, the femur is moved from a neutral position or full extension of the leg in small steps upwards while being moved around the center of rotation located in the pelvis. The pelvis, within which the femur can turn, is stationary with respect to the mentioned stationary reference. Thus, the distal femur axis end point and consequently the femur is in a different position each time a positional information value of the femur is acquired.

b) the at least four different acquired positional information values of the femur are used to determine the position of the center of rotation of the femoral head around which the femur was moved in relation to a femur reference. The femur reference can be any reference having a fixed or stable or almost fixed relationship with respect to the femur and is according to a preferred embodiment non-invasively attached to the femur. The femur reference can be a reference being attached or connected to a plate which optionally can be attached or adhered to the outside of the thigh without using pins or the like (so-called pinless plate) being described below and being attached to the upper leg using for example an adhesive foil or a Velcro®. Since at least four different positions of the femur are taken into consideration, the center of rotation around which the femur was moved, can easily be determined, since the surface of a sphere on which the femur reference is moved around the center of rotation can be determined when knowing at least four different points being located on the surface of this sphere. Of course, more than four different positional information values can be acquired and/or used for determining the center of rotation.

c) A femur information defining the position and/or orientation of the femur is acquired by detecting via a hand-held device, such as a hand-held camera, e.g. a mobile telephone or a tablet computer comprising a built-in camera, a femur reference and at least one further information described later.

d) Using the femur information and the at least one further information, the distal end point of the femur axis and the proximal end point of the tibia axis are determined at least in relation to the femur reference.

e) the distal end point of the tibia axis can be determined by acquiring via the hand-held device the positional information of an ankle reference being located at the distal end point of the tibia axis. The ankle reference is preferably a reference being non-invasively attached to the ankle to be in a preferably stable and fixed relationship with respect to the ankle. Thus, detecting the ankle reference means detecting the distal tibia axis end point. Preferably the shape and/or dimensions of the ankle reference, especially the positional relationship between ankle reference markers and the attachment portion of the ankle reference is known, so that a predefined relationship between the ankle reference markers and the tibia axis end point exists when the ankle reference is properly attached to the ankle. For example, the tibia axis end point can be assumed to be the midpoint between the contacting points. The above principles may also supply to other references mentioned herein, for example a femur reference.

Thus, using the above described data processing method, the center of rotation of the femoral head, the distal femur axis end point considered to be the same point as the proximal tibia axis end point and the distal tibia end point can be determined as three points defining the mechanical femur axis and the mechanical tibia axis, without using reference arrays invasively attached to the patient's bones. It is possible to obtain this information using only reference arrays which are non-invasively attached to the patient, e.g. using an adhesive foil.

The navigation can be performed with a single device being for example a single hand-held device that tracks markers, preferably optical markers, by using a built-in video camera. The registration of the anatomy can thus be performed without using any further device, such as a pointer device. The only required equipment is a hand-held video camera which does not need to be sterile, thus, reducing the costs compared to known methods requiring the employment of navigation systems with expensive stereo cameras.

The at least one further information acquired in above described step a) is at least or exactly one of the following:
the detection of a femur reference, e.g. a marker or marker arrangement connected to an attaching element, such as an adhesive foil, being positioned at the outer surface or skin of the leg.
the detection of a femur reference and an epicondylar contacting device.
the detection of the position of the tip of a pointer device to which the hand-held device being for example a hand-held camera, is attached, wherein the tip is placed on one of the femoral epicondyles.

The at least one further information of above described step c) is at least or exactly one of the following:
detecting an ankle reference for at least three different degrees of flexion of the tibia in relation to the femur, wherein the tibia is in a different position with respect to the femur each time a positional information value of the flexion is acquired.
detecting the position of epicondylar contacting devices.
detecting the position of the tip of a pointer device to which the hand-held device being preferably a hand-held camera is attached, in relation to a femur reference, the tip being placed on one of the femoral epicondyles.

According to an embodiment, in the above step d), the distal end point of the femur axis and the proximal end point of the tibia axis are also determined in relation to the ankle reference.

According to a further embodiment, an additional landmark is acquired in step c) to refine the determination of the distal end point of the femur axis and the proximal end point of the tibia axis in step d).

According to a further embodiment, the additional landmark is in the middle of the patella.

According to a further aspect, the invention is directed to a method for determining the varus/valgus angle and/or the flexion angle of a leg using the positional information of main components of a leg, such as for example the mechanical femur axis and the mechanical tibia axis or the respective end points, being determined as described above. The method for determining the varus/valgus angle and/or the flexion angle comprises the following steps performed by a computer:

f) detecting at least one femur reference and an ankle reference via a hand-held device, preferably a hand-held camera, and acquiring positional information of the femur reference and the ankle reference. The above-mentioned epicondylar contacting devices can also be considered as being a femur reference. The femur reference in general should be attached permanently or temporarily to be in a fixed or almost fixed relationship to the femur and preferably has a predefined known positional relationship to the epicondyles. The leg is preferably in a neutral position or full extension when this acquiring step is performed.

g) the position of the center of rotation of the femoral head is determined using the relation to the femur reference determined in step b).

h) the position of the distal end point of the femur axis is determined used at least the relation to the femur reference determined in step d).

i) the position of the femur axis is determined as being the axis connecting the center of rotation of the femoral head and the distal end point of the femur axis.

j) the position of the proximal end point of the tibia axis is determined using at least the relation of the femur reference determined in step d).

k) the position of the distal end point of the tibia axis is determined as being the positional information of the ankle reference.

l) the position of the tibia axis is determined as being the axis connecting the position of the proximal end point of the tibia axis and the distal end point of the tibia axis.

m1) the varus/valgus angle is determined as the angle between the femur axis and the tibia axis in the frontal plane.

m2) according to an alternative or an additional step, the flexion angle is determined as the angle between the femur axis and the tibia axis in the sagittal plain.

According to a preferred embodiment, the femur reference is a plate being attached non-invasively to the femur, such as a plate, preferably a so-called pin-less plate provided by Brainlab, comprising markers. The femur reference can be attached to the femur using an adhesive foil or a Velcro®. According to an alternative or further embodiment, the femur reference can be one or more epicondylar connecting devices.

The ankle reference can preferably provide information about the location of the distal tibia axis end point, preferably when connected in a fixed and known relationship to the distal tibia axis end point.

According to a further embodiment, the flexion/extension (slope) angle can be navigated during tibia osteotomies. If the anterior/posterior (AP)-direction is determined during the registration process, it is possible to navigate the flexion/extension (slope) angle of the mechanical femur and tibia axis. The registration can work in several ways:
flexion movement of tibia against femur
holding a pointer device in AP-direction and registering this orientation
determining a plane, using the epicondyles and one other landmark, that is oriented in a certain angle (for example 90°) to the AP-direction
design a placement of one reference array in such a way that the AP-direction is known through the hardware.

For navigation of the flexion angle, the same actions and references are required as for the above-mentioned varus/valgus navigation.

The concepts above can be applied for tibia osteotomies. For navigation of femur osteotomies, they can be adapted appropriately. All registration processes can remain the same.

### BRIEF DESCRIPTION OF THE DRAWINGS

The invention will be described in more detail by reference to an exemplary embodiment:

FIG. 1 shows leg to illustrate the position of the femur axis and the tibia axis;

FIG. 2 shows an arrangement according to a first concept of the invention;

FIG. 3 shows an arrangement according to a second concept of the invention;

FIG. 4 shows an arrangement according to a third concept of the invention;

FIG. 5 shows the movement of the tibia into different positions to register the knee joint center;

FIG. 6 shows a first embodiment of a non-invasive femur reference; and

FIG. 7 shows a second embodiment of a non-invasive femur reference.

### DETAILED DESCRIPTION OF THE INVENTION

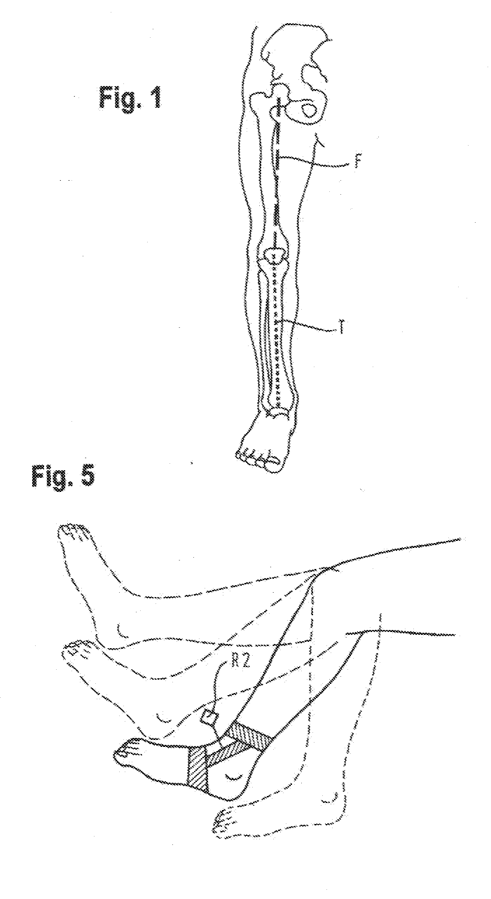

FIG. 1 shows a lower extremity or leg with a mechanical femur axis F shown as dashed line and the tibia axis T shown as dotted line. In a healthy leg, the femur axis and the tibia axis are in line with each other (0° varus/valgus).

### Concept 1

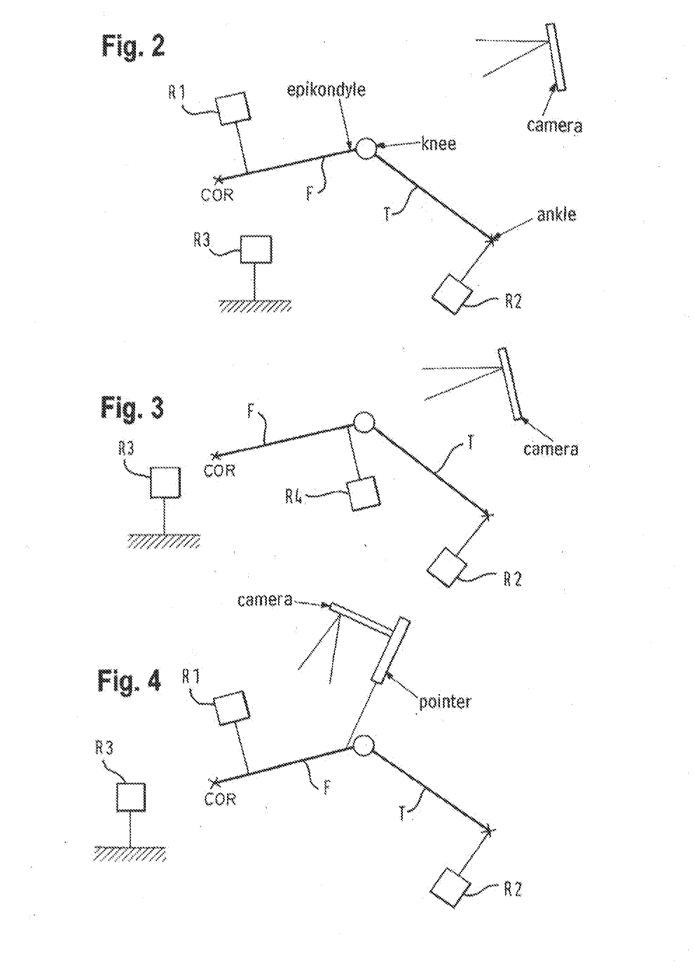

FIG. 2 shows an arrangement of a first concept of the present invention.

A hand-held camera is provided as a navigation device. All subsequently mentioned references R1, R2, R3, R4 should be positioned or located to be in the field of view of the camera, at least when acquiring respective position data. References R1 to R4 are markers or marker devices or reference stars as defined above.

A fixed reference R3 can for example be attached to the table on which the patient's leg having the shown femur axis F and tibia axis T connected by the knee is positioned. The femur or femur axis F and tibia or tibia axis T can be moved with respect to the fixed reference R3, whereas the center of rotation COR of the femur axis is in a stable and fixed relationship with respect to the fixed reference R3.

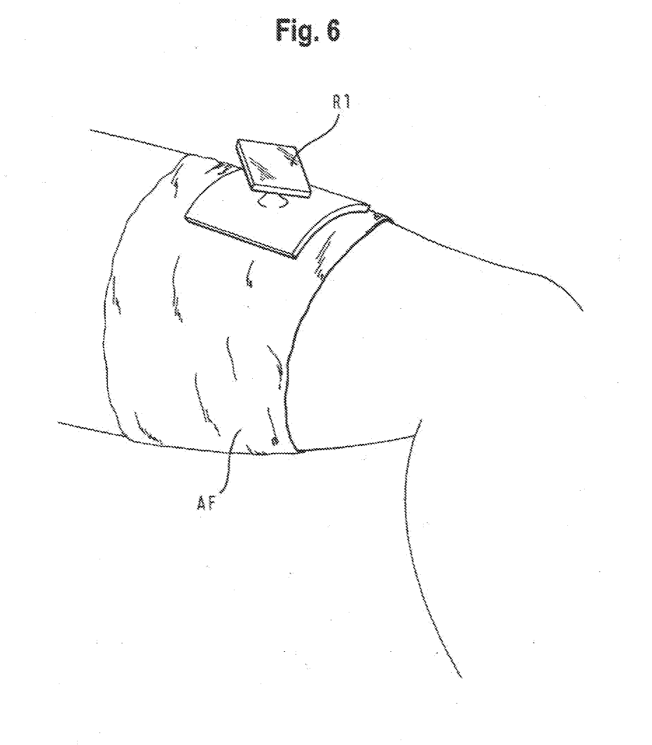

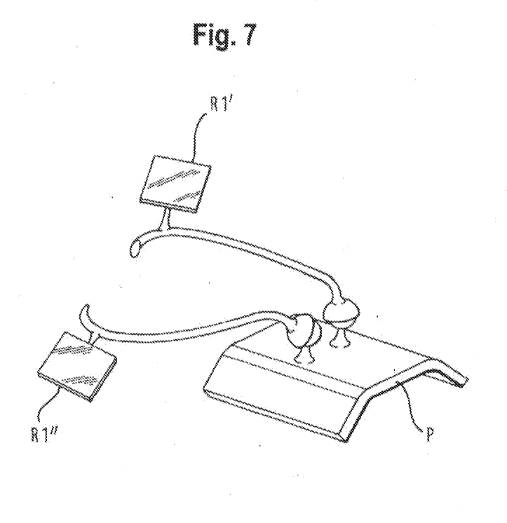

A femur reference such as the above-mentioned plate, preferably a so-called pin-less plates or any other reference R1 being non-invasively attached to the femur, such as the references shown in FIGS. 6 and 7, is attached to the femur F, for example using an adhesive foil. Although shown in FIGS. 2 to 4 for illustrative purposes, the respective references R1, R2 and R4 are not directly attached to the femur (axis) F or the tibia (axis) T. Instead, those references are attached non-invasively to the outside of the respective anatomical structure so as to have a preferably fixed and pre-defined positional relationship with respect to the respective axis F or T or the end points.

An ankle reference R2 is attached to the ankle or distal tibia axis end point in such a way that it provides information about the location of the distal tibia axis end point.

To register the center of rotation COR of the femoral head, the fixed reference R3 and the reference R1 on the femur F being for example the mentioned plate, preferably a so-called pin-less plate for the markers, is required. The femur F is moved or placed in at least four different positions. The navigation device being the hand-held camera is held such that the fixed reference R3 and the femur reference R1 is in the field of view of the camera.

Once the at least four different positions of the femur reference R1 connected or related to the femur being placed in at least four different positions is acquired, the center of rotation COR can be determined and registered in relation to the femur reference R1. In other words, a positional relationship between the femur reference R1 and the center of rotation COR can be calculated.

To register the distal femur axis end point being the proximal tibia axis end point, the femur reference R1 and the ankle reference R2 are required.

The tibia is placed in at least three different degrees of flexion in relation to the femur, as for example shown in FIG. 5 which illustrates four different degrees of flexion in relation to the femur to increase accuracy.

The navigation device being the hand-held camera is held in such a way that the camera has both references, the femur reference R1 and the tibia reference R2, in the field of view.

As a result, the distal femur axis end point and the proximal tibia axis end point can be calculated and registered in relation to the femur reference R1 and the tibia reference R2.

The registration of the distal tibia axis end point (corresponding to above step e)) uses the tibia or ankle reference R2. Once the hand-held camera detects the reference, the distal tibia axis end point is known and thus registered. The leg is preferably placed in a neutral position or full extension.

To navigate the axis (varus/valgus angle), the femur reference R1 and the ankle reference R2 is required. The leg is placed in a neutral position, such as full extension. The varus/valgus angle can be determined and optionally be displayed as being the angle between the mechanical femur and tibia axes in the frontal plane. In a similar manner, the flexion angle being the angle between the mechanical femur and tibia axes in the sagittal plane can be determined.

According to the first concept, the mid-point of the knee is determined by placing the tibia and the femur into three different positions relative to each other, i.e. into at least three different degrees of flexion. The rotation point of this movement can be determined. However, in case of a severe varus or valgus leg, the point that is determined will not lie in the middle of the knee joint because the mechanical axis of the leg does not pass through that joint. Therefore, according to a preferred embodiment, an additional landmark is acquired, which additional landmark can for example be in the middle of the patella. Using this additional landmark, the determined rotation point can be transferred to its correct location in relation to the leg.

### Concept 2

According to a second concept of the invention, which is illustrated in FIG. 3, contacting devices R4 for identification of the femoral epicondylar points are attached to a non-invasive reference being attached to the femur F.

Furthermore, a fixed reference R3 and an ankle reference R2 are provided as described above for FIG. 2.

To register the center of rotation COR of the femoral head, the fixed reference R3 and the femur reference R4 is required.

The femur F is placed in at least four different positions and the hand-held navigation device or camera is held to have both references R3 and R4 in the field of view.

As a result, the center of rotation COR can be registered in relation to the femur reference R4.

To register the distal femur axis end point being the proximal tibia axis end point, the femur reference R4 is required. The leg is placed in a neutral position, such as full extension. Whenever the hand-held camera detects both references R2 and R4, the femoral epicondylar points are registered. The distal femur axis end point can be calculated from the detected reference R4, for example by calculating the midpoint between the contacting points of the femur reference R4 (epicondyle points). The proximal tibia axis end point is assumed to be the same point.

To register the distal tibia axis end point, the ankle reference R2 is required.

The registration of the distal tibia axis end point (corresponding to above step e)) uses the tibia or ankle reference R2. The leg is placed in a neutral position or full extension. Once the hand-held camera detects the reference, the distal tibia axis end point is known and thus registered.

The navigation of the axes (varus/valgus angle and/or flexion angle) requires the femur reference R4 and the ankle reference R2.

The leg is placed in a neutral position, such as for example full extension. The varus/valgus angle and/or the flexion angle between the femur and tibia axes can be determined whenever the hand-held camera detects both references R2 and R4.

### Concept 3

According to a third concept of the invention being illustrated in FIG. 4, a fixed reference R3, a femur reference R1 and an ankle reference R2 is provided.

In addition, a pointer device with an attachment unit for the navigation device being for example the hand-held camera, is provided.

To register the center of rotation of the femoral head, the fixed reference R3 and the femur reference R1 are required.

The camera is attached or positioned to the hand-held pointer device. The pointer tip is placed on one of the femoral epicondyles and kept there while the femur F is placed in at least four different positions.

The pointer with the attached navigation device is held or turned in such a way that the fixed reference R3 is in the field of view.

As a result, the center of rotation COR can be registered in relation to the fixed reference R3.

The registration can then be transferred to the femur reference R1 after the registration of the epicondylar points in the neutral leg position.

To register the distal femur axis end point being the proximal tibia axis end point, the femur reference R1 is required.

The leg is placed in a neutral position, such as full extension.

The tip of the pointer is placed on each femoral epicondyle in turn and each landmark is registered.

As a result, the femoral epicondylar points are registered in relation to the femur reference R1.

The distal femur axis end point can be calculated from this information.

The proximal tibia axis end point is assumed to be the same point.

The registration of the distal tibia axis end point (corresponding to above step e)) uses the tibia or ankle reference R2. The leg is placed in a neutral position or full extension. Once the hand-held camera detects the reference, the distal tibia axis end point is known and thus registered.

To navigate the axis (varus/valgus or flexion angle), the femur reference R1 and the ankle reference R2 are required.

The leg is placed in a neutral position, such as full extension.

Whenever the camera detects both references R1 and R2, the points are known, so that the varus/valgus and flexion angle between the mechanical femur and the tibia axis can be displayed.

### Navigation of Axis Passing Point in Knee

According to several publications (e.g. Fujisawa Y, Masuhara K, Shiomi S. The effect of high tibial osteotomy on osteoarthritis of the knee. An arthroscopic study of 54 knee joints. Orthop Clin North Am 1979; 10(3): 585-608.) the mechanical leg axis does not necessarily pass through the center of the knee after HTO surgery. The desired passing point is given as percent of the distance from either medial to lateral cortex or middle of the knee to the cortex.

This information can also be provided by the navigation device, if the positions of the medial and lateral cortex of the tibia the medial and lateral epicondyles are known (concepts 2 and 3).

FIG. 6 shows an embodiment of a femur reference R1 having a reflective surface being attached to the upper leg or femur by means of an adhesive foil AF.

FIG. 7 shows a further embodiment of a femur reference R1 having two reflective elements R1′ and R1″ being connected to plate P which can be attached to the upper leg or femur, e.g. by means of an adhesive foil AF.

1-12. (canceled)

13. A navigation system for computer-assisted surgery comprising a computer having a processor configured to execute a computer-implemented method for determining a positional information of characteristic points of a leg, the method comprising executing, on the processor of the computer, steps of:
a) acquiring, at the processor, by detecting via a hand-held device, a position of a stationary reference and epicondylar contacting devices or detecting a position of a tip of a pointer device to which the hand-held device or a hand-held camera is attached, the tip being placed on one of the femoral epicondyles, at least four different positions of the femur, wherein the pelvis within which the femur can turn is stationary with respect to the position of the stationary reference and the femur is in a different position each time a positional information value of the femur is acquired;
b) determining, by the processor, from the at least four different acquired positional information values of the femur the position of the center of rotation of the femoral head in relation to the position of a femur reference;
c) acquiring, at the processor, a femur information by detecting via the hand-held device the position of the femur reference, and the position of the tip of the pointer device to which the hand-held device or hand-held camera is attached in relation to the femur reference, the tip being placed on one of the femoral epicondyles;
d) determining, by the processor, from the femur information and the position of the tip of the pointer device acquired in step c) the distal end point of the femur axis and the proximal end point of the tibia axis at least in relation to the position of the femur reference; and
e) determining, by the processor, the distal end point of the tibia axis by acquiring via the hand-held device the positional information of an ankle reference being at the distal end point of the tibia axis.

14. The navigation system of claim 13 comprising the hand-held device or mobile detection device for detecting the position of references to generate detection signals and to supply the generated detection signals to the computer, such that the computer can determine the position of the references and/or elements connected thereto on the basis of the detection signals received.

15. The navigation system of claim 13, wherein the processor is configured to execute the steps of:
f) acquiring, at the processor, by detecting at least one femur reference and the ankle reference via the hand-held device, the positional information of the femur reference and the ankle reference;
g) determining, by the processor, the position of the center of rotation of the femoral head using the relation to the femur reference determined in step b);
h) determining, by the processor, the position of the distal end point of the femur axis using at least the relation to the femur reference determined in step d);
i) determining, by the processor, the position of the femur axis as being the axis connecting the center of rotation of the femoral head and the distal end point of the femur axis;
j) determining, by the processor, the position of the proximal end point of the tibia axis using at least the relation to the femur reference determined in step d);
k) determining, by the processor, the position of the distal end point of the tibia axis as being the positional information of the ankle reference;
l) determining, by the processor, the position of the tibia axis as being the axis connecting the position of the proximal end point of the tibia axis and the distal end point of the tibia axis;
m1) determining, by the processor, the varus/valgus angle as the angle between the femur axis and the tibia axis in the frontal plane and/or
m2) determining, by the processor, the flexion angle between the femur axis and the tibia axis in the sagittal plane.

16. A non-transitory computer-readable program storage medium storing a program which, when executed by a processor of a computer, causes the processor to execute a computer-implemented method for determining the positional information of characteristic points of a leg, the method comprising executing, on the processor of the computer, steps of:
a) acquiring, at the processor, by detecting via a hand-held device, a position of a stationary reference and epicondylar contacting devices or detecting a position of a tip of a pointer device to which the hand-held device or a hand-held camera is attached, the tip being placed on one of the femoral epicondyles, at least four different positions of the femur, wherein the pelvis within which the femur can turn is stationary with respect to the position of the stationary reference and the femur is in a different position each time a positional information value of the femur is acquired;
b) determining, by the processor, from the at least four different acquired positional information values of the femur the position of the center of rotation of the femoral head in relation to the position of a femur reference;
c) acquiring, at the processor, a femur information by detecting via the hand-held device the position of the femur reference, and the position of the tip of the pointer device to which the hand-held device or hand-held camera is attached in relation to the femur reference, the tip being placed on one of the femoral epicondyles;
d) determining, by the processor, from the femur information and the position of the tip of the pointer device acquired in step c) the distal end point of the femur axis and the proximal end point of the tibia axis at least in relation to the position of the femur reference; and
e) determining, by the processor, the distal end point of the tibia axis by acquiring via a hand-held device the positional information of an ankle reference being at the distal end point of the tibia axis.

17. A computer-implemented method for determining the positional information of characteristic points of a leg, the method comprising executing, on a processor of a computer, steps of:
a) acquiring, at the processor, by detecting via a hand-held device, a position of a stationary reference and epicondylar contacting devices or detecting a position of a tip of a pointer device to which the hand-held device or a hand-held camera is attached, the tip being placed on one of the femoral epicondyles, at least four different positions of the femur, wherein the pelvis within which the femur can turn is stationary with respect to the position of the stationary reference and the femur is in a different position each time a positional information value of the femur is acquired;
b) determining, by the processor, from the at least four different acquired positional information values of the femur the position of the center of rotation of the femoral head in relation to the position of a femur reference;
c) acquiring, at the processor, a femur information by detecting via the hand-held device the position of the femur reference, and the position of the tip of the pointer device to which the hand-held device or hand-held camera is attached in relation to the femur reference, the tip being placed on one of the femoral epicondyles;
d) determining, by the processor, from the femur information and the position of the tip of the pointer device acquired in step c) the distal end point of the femur axis and the proximal end point of the tibia axis at least in relation to the position of the femur reference (R1); and
e) determining, by the processor, the distal end point of the tibia axis by acquiring via the hand-held device the positional information of an ankle reference (R2) being at the distal end point of the tibia axis.

18. The method according to claim 17, wherein in step d) the distal end point of the femur axis and the proximal end point of the tibia axis are also determined in relation to the ankle reference (R2).

19. The method according to claim 17, wherein an additional landmark is acquired in step c) to refine the determination of the distal end point of the femur axis and the proximal end point of the tibia axis in step d).

20. The method according to claim 19, wherein the additional landmark is in the middle of the patella.

21. The method of claim 17 further comprising the steps:
f) acquiring, at the processor, by detecting at least one femur reference and the ankle reference via the hand-held device, the positional information of the femur reference and the ankle reference;
g) determining, by the processor, the position of the center of rotation of the femoral head using the relation to the femur reference determined in step b);
h) determining, by the processor, the position of the distal end point of the femur axis using at least the relation to the femur reference determined in step d);
i) determining the position of the femur axis as being the axis connecting the center of rotation of the femoral head and the distal end point of the femur axis;
j) determining, by the processor, the position of the proximal end point of the tibia axis using at least the relation to the femur reference determined in step d);
k) determining, by the processor, the position of the distal end point of the tibia axis as being the positional information of the ankle reference;
l) determining, by the processor, the position of the tibia axis as being the axis connecting the position of the proximal end point of the tibia axis and the distal end point of the tibia axis;
m1) determining, by the processor, the varus/valgus angle as the angle between the femur axis and the tibia axis in the frontal plane and/or
m2) determining, by the processor, the flexion angle between the femur axis and the tibia axis in the sagittal plane.

22. The method according to claim 17, wherein:
the femur reference is a plate comprising markers, attached to the femur using an adhesive foil and/or epicondylar contacting devices;
the ankle reference provides information about the location of the distal tibia axis end point;
the hand-held device is a hand-held camera.
